(19) United States
(12) Patent Application Publication
(10) Pub. No.: US 2009/0018486 A1
(43) Pub. Date: Jan. 15, 2009

(54) Diagnosis and treatment of vericocele and prostate disorders

(76) Inventors: Menachem Goren, Petach-Tikva (IL); Yigal Gat, Ramat-Gan (IL)

Correspondence Address: MARTIN D. MOYNIHAN d/b/a PRTSI, INC., P.O. BOX 16446, ARLINGTON, VA, 22215, US

(21) Appl. No.: 11/826,283

(22) Filed: Jul. 13, 2007

(51) Int. Cl. A61M 25/10; A61N 1/30

(52) U.S. Cl. 604/21; 128/898; 600/300; 604/164.03; 604/509; 604/96.01

(57) ABSTRACT

A method for forestalling or therapy, at least partially, of BPH or prostate cancer comprising identifying a reflux of venous blood to the prostate and impeding the reflux by treating veins effecting or conveying the reflux, and related apparatus and kit.

### FIELD OF THE INVENTION

The invention relates to diagnosis and/or treatment of varicocele, benign prostate hyperplasia (BPH), prostate cancer and/or disorders of testosterone hormone. Some embodiments relate to the diagnosis and treatment to impaired testicular venous drainage.

### BACKGROUND OF THE INVENTION

Deterioration of the one-way valves in the internal spermatic veins, clinically manifested as varicocele, may lead to reduced drainage, or even a reflux, of venous blood into the testes.

The left internal spermatic vein enters the left renal vein at a right angle near a potential site of compression by the superior mesenteric artery, while the right spermatic vein drains at an acute angle into the inferior vena cava. These anatomical factors, and the additional effect of gravity, promote backflow of blood in the left internal spermatic vein (more so than the right spermatic vein). Consequently, varicocele of the left internal spermatic vein can be diagnosed relatively easily, and has been widely linked to male infertility in the medical literature. See, for example, Gorelick J L, Goldstein M 1993 Loss of infertility in men with varicocele, Fertility and Sterility 59, 613-616; Greenberg S H (1977) Varicocele and male fertility, Fertil Steril 28(7),699-70.

More recently, varicocele of the right internal spermatic vein was recognized to play a similar role in male infertility. See, for example, Gat Y, Bachar G N, Zukerman Z and Gornish M (2004) Varicocele: a bilateral Disease, Fertil Steril 81,424-42.

Studies over the past years demonstrated a correspondence between varicocele and serum testosterone level, though the findings did not converge to a consistent and plausible correlation. See, for example, Gat Y, Gornish M, Belenky A and Bachar G N, Elevation of serum testosterone and free testosterone after embolization of the internal spermatic vein for the treatment of varicocele in infertile men, Human Reproduction Vol. 19, No. 10 pp. 2303-2306, 2004.

Though varicocele was connected somehow with testosterone level, and testosterone is known for a long time to play a role in prostate cancer (for example, Campbell's Urology (ed-in-chief Walsh, P.) 1245-1249, 77, 2566 (Saunders Eight Edition, Philadelphia, USA, (2002)), there was no established causal correlation between varicocele and prostate disease, and, paradoxically, relatively low levels of serum testosterone were found in patients with prostate cancer (see, for example, Raivio T, Santti H, Schatzl G, Gsur A, Haidinger G, Palvimo J J, Janne O A, Madersbacher S. Reduced circulating androgen bioactivity in patients with prostate cancer. Prostate 2003;15:194-8).

A similar paradox was also found with respect to BPH. See, for example, Roberts R O, Jacobson D J, Rhodes T, Klee G G, Leiber M M, Jacobsen S J. Serum sex hormones and measures of benign prostatic hyperplasia. Prostate. Oct. 1, 2004; 61(2):124-31).

Part of the relevant anatomy is schematically illustrated in FIG. 1 and FIG. 2.

FIG. 1 schematically illustrates a typical testicular and prostate venous drainage system of a human male. One drainage path from a testes 104 comprises the pampiniform plexus 118 to the internal spermatic vein 102 that leads towards the inferior vena cava 106 through one-way valves 108. Normally, the valves 108 facilitate venous blood flow upwards towards the vena cava 106, and inhibit back flow down to the testes 106.

Another drainage path comprises a sequence of pampiniform plexus 118 to the deferential vein 110, the vesicular vein 112, the internal iliac vein 114, the common iliac vein 116 towards the inferior vena cava 106. The latter path is shared by the prostate 120 drainage path from the vesicular plexus 128 towards the vesicular vein 112 and onwards.

Arteries 122 supply arterial blood to the microcirculation 124 of prostate 120 and the microcirculation 126 of testes 104.

FIG. 2 schematically illustrates typical testicular and prostate venous drainage paths in a normal left side of a human male where the arrows directions illustrate the venous blood flow as described above.

Since the one-way valves 108 in the internal spermatic vein 102 block back flow down to the testes 104, they isolate hydrostatic pressure from the sections between them, so that a typical pressure at the entry 142 to the left internal spermatic vein 102 is about 5-6 mmHg and may be somewhat lower at entry 144 to the right spermatic vein 130.

### SUMMARY OF THE INVENTION

A broad aspect of some embodiments of the invention relates to the innovative recognition that impaired valves in the spermatic veins play a causative role in prostate and testosterone disorders, such as BPH, cancer and/or aging, possibly as outlined below. Without being bound to a particular theory, preliminary treatments based on its presumptions have yielded positive results, which potentially validate the practical methods derived from the theory.

Internal spermatic veins with incompetent or destroyed one way valves cannot maintain upstream venous flow, resulting in a blood column of about 35-40 cm with relatively elevated hydrostatic pressure. The elevated hydrostatic pressure prevents venous blood in the pampiniform plexus (which drains the testis) from flowing upwards to the inferior vena cava on the right side and towards the renal vein on the left side. Rather, under these conditions, venous blood from the testis is diverted, via the deferential vein and the vesicular plexus, into the prostatic venous plexus in and around the prostate.

Since, under these conditions, the hydrostatic pressure in the internal spermatic vein and pampiniform plexus, and hence the venous pressure (due to connected vessels phenomenon) within the smaller veins, may exceed the arteriolar pressure in the testicular and prostate microcirculation, the supply of arterial blood to these organs is disrupted (resulting in hypoxia). The disrupted venous drainage and arterial supply lead to blood stagnation and degenerative processes in the seminiferous tubules- the sperm production site, resulting in male infertility. See, for example, Gat Y., Chakraborty, J., Zukerman, Z., Gornish, M. Varicocele, Hypoxia, and Male Infertility, Fluid mechanics analysis of the impaired testicular venous drainage system. Hum. Reprod (2005); 20:2614-2619. Editorial Comment in J. Urol. (2006), April 17(4), 1454).

The altered venous flow diverts venous blood from the testis towards the prostate in elevated hydrostatic pressure and restricts the drainage of prostate veins, possibly leading to swelling (dilation) of the prostate.

Furthermore, under normal physiologic condition, free testosterone (secreted by the testes) drains to the general blood circulation where it is diluted and binds (about 98%) to proteins such as SHBG (serum hormone binding globulin) and albumin. With abnormal (or damaged) internal spermatic vein valves, however, the reverse flow diverts free testosterone from its production site in the testis directly to the prostate, greatly increasing the concentration of testosterone in the gland, and particularly free testosterone, to an excessive level far above normal levels (typically beyond the normal level of about 17 nmol/l and 10 nmol/l of free testosterone) It is theorized that the excessive level of free testosterone in the prostate stimulates cell proliferation (such as BPH) and/or cancer.

Though varicocele and venous backflow were known for long time to have some effects on the testis and fertility, the mechanisms outlined above and their effects on the prostate comprise, at least partially, the new theory mentioned earlier proposed by the inventors.

An aspect of exemplary embodiments of the invention relates to a method for forestalling and therapy of BPH and prostate cancer by preventing or impeding the reflux of venous blood (rich in free testosterone) to the prostate.

In exemplary embodiments of the invention, the reflux is prevented or impeded by occlusion, of the internal spermatic vein, or veins, that have effected the hydrostatic pressures and back flow (e.g. by embolization, sclerosis, or occlusion). Optionally or alternatively, some or all veins through which the reflux flows to the prostate are occluded. Optionally or additionally, bypass veins 136 that might have developed (for example, as a result of the hydrostatic pressure) are occluded if they carry blood from the testis to the prostate. Optionally, the veins carrying blood to the prostate, such as the deferential vein, are occluded initially in order to prevent testicular venous blood rich in testosterone (relative to normal circulation) from reaching the prostate, allowing recovery from the venous congestion and shrink the swelling and/or tumor. Optionally, a spermatic vein which affected the excessive hydrostatic pressure is occluded, optionally after treatment of veins leading blood to the prostate, to allow drainage of testosterone rich blood to the body via vessels such as the scrotal veins or other bypass veins 136. Optionally, the occlusion is applied in the opening of a vein or at a spot or region along the vein so that blood cannot flow in the vessel.

In exemplary embodiments of the invention, the occlusion is carried out by injecting, for example, by a catheter, a sclerosant into the vein. Optionally, other methods and/or elements are used, such as placement of coils or silk that block the vein passage and/or induce thrombosis. Optionally, hot liquid or contrast medium is injected that effect shrinkage and occlusion of the vessel. Optionally, other methods are used such as ablation.

An aspect of some embodiments of the invention relates to using sclerosants and/or blocking elements and/or sclerotic medication for forestalling and/or treating BPH and/or prostate cancer.

In exemplary embodiments of the invention, persons diagnosed for varicocele by standard procedures (e.g. visual or diagnostic imaging procedures) are selected for prostate hypertrophy diagnosis. Optionally, if hypertrophy is present in a certain extent, the person is treated as described, taking into account that the treatment such as occlusion may affect fertility, optionally positively, as the hydrostatic pressure is reduced and testosterone may pass up-flow from the testis. Optionally, the treatment has anti-aging effects as testosterone is free to drain from the testis to the bloodstream, raising testosterone concentration in the blood when the hydrostatic pressure is reduced. Optionally, the invention allows to treat patients in a priority to the significance of the prostate malfunction.

In exemplary embodiments of the invention, the treatment is potentially applicable in forestalling by either (a) treating varicocele as described above, preventing the development of cancer, and hence, metastases, or (b) if cancer is already present, occlusion of at least the deferential vein or other vessels that drain from the prostate to the blood stream, trying to prevent cancerous cells from leaking from the prostate to the blood stream.

In exemplary embodiments of the invention, a sclerosant is used for the manufacture of a medicament for forestalling and/or treating BPH or prostate cancer in a subject. Optionally, the sclerosant is adapted to treating backflow that effects BPH and/or prostate cancer. Optionally, the adaptation comprises the composition of materials and/or their proportions.

In exemplary embodiments of the invention, a medication such as an antigen bound guided molecular therapy, optionally as a medication, may be used as part of the treatment.

An aspect of some embodiments of the invention relates to a method for diagnosis, or estimation, of the degree of valves degradation and/or venous reflux to the prostate and/or varicocele by a palpation, or any other way of estimating prostate situation, of the prostate for hypertrophy.

Optionally, the diagnosis relates to effects from either a unilateral (left or right side) malfunction or bilateral (left and right side), or a combination of malfunction levels of each side. Optionally, the malfunctioning side may be identified by methods such as radiology or ultrasonography.

In exemplary embodiments of the invention, reflux may be diagnosed by injecting contrast medium or the appropriate diagnostic radionuclide agent into suspected regions and following its dispersion, or motion, by x-ray or gamma camera. Optionally, other diagnostic methods for blood flow analysis may be used ultrasonography.

An aspect of some embodiments of the invention relates to a method for diagnosis of varicocele and/or venous reflux by measuring venous testosterone levels between testes and the prostate where reflux is expected, or a region before testicular venous blood enters the vena cava. Optionally, the level of free and/or bound (serum) testosterone is measured. Optionally, testosterone may be tested in the arterial blood. Optionally and additionally, the tests are made when the patient is standing, and then when he is lying down (reducing the hydrostatic pressure), checking if the testosterone level reduced, which may indicate the presence of varicocele or malfunction of the spermatic vein valves and consequent prostate malfunction.

Optionally, the diagnosis is related to or based on the measurement location and/or proximity to the testes and/or the prostate.

An aspect of some exemplary embodiments of the invention relates to a method for reducing the expression or symptoms of male aging due to deficient serum testosterone due to backflow to the prostate, by increasing testosterone supply to the blood stream.

In exemplary embodiments of the invention, aging symptoms or expressions are reduced, to some extent, by preventing reflux in the internal spermatic veins, resulting in restoration of normal blood circulation in the testes. Optionally, the testosterone level is increased due to changing blood circulation pathways.

Optionally, aging expressions or symptoms are reduced due to one or both of (a) at least a partial restoration of adequate drainage of the testis venous blood to the systemic blood circulation, allowing testosterone to drain to the bloodstream and increase the testosterone bound and unbound concentration, and (b) at least partial allowance of fresh arterial blood to the testis, healing, at least partially, the congested testis. The fresh arterial supply and restoration of drainage increases testosterone production due to either (a) supply of oxygen and removal of carbon dioxide and waste material, and (b) reducing an effect of a feed back cycle that might have signaled the testes to reduce testosterone production when it was present in high concentrations, preserving testosterone production capacity.

In exemplary embodiments of the invention, the reflux is prevented or impeded by occlusion, for example, as described above (with respect to BPH and cancer).

Optionally, testosterone, as an injection, patch or orally administered, may given to the patient to test if beneficial effects are expected to achieved before occlusion by elevating the blood testosterone levels therapeutically. Optionally or additionally, the vessels leading venous blood to the prostate may be occluded and the resultant effects evaluated. Optionally, the spermatic veins, which may effected the reflux, are occluded. Optionally, one side (left or right) is treated (in order to preserve functioning valves), and the results are evaluated if further treatment is required.

An aspect of some exemplary embodiments of the invention relates to a mechanism designed to pass through venous valves.

In exemplary embodiments of the invention, the mechanism is guide-wire for an intravascular catheter. Optionally the mechanism comprises a catheter. Optionally, the guide-wire is used to guide a catheter though a valve. Optionally, the catheter is intended for sclerotherapy.

In exemplary embodiments of the invention, the guide-wire comprises, near the distal end, an expandable element which may be retracted back. By expanding the element near a valve the vein is widened, opening the valve orifice. consequently, the expanded elements is contracted and the guide-wire is inserted through the valve orifice before the valve closes.

In exemplary embodiments of the invention, the element comprises a wire mesh. Optionally, it comprises other expandable and contractible mechanisms, such as an inflatable balloon.

An aspect of some exemplary embodiments of the invention relates to an intravascular catheter for sclerotherapy, designed to apply the sclerosing agent into an intended region of a blood vessel limiting, at least to some extent, the agent flow or drainage to another region (which flow may be detrimental).

In exemplary embodiments of the invention, the catheter comprises two (or more) inflatable balloons and one or more apertures defined in the catheter between the balloons. By placing the catheter so that the balloons are about the ends of a vessel section to be occluded and inflating the balloons into the vein walls, the section is blocked and a sclerosing agent may be injected through the holes while the balloons impede, or block, a drain or flow beyond the section. Optionally, the catheter is designed for use with the guide-wire described above.

An exemplary embodiment of the invention comprises a method for forestalling and/or therapy, at least partially, of BPH and/or prostate cancer comprising:

(a) identifying a reflux of venous blood to the prostate; and

(b) impeding the reflux by treating veins effecting and/or conveying the reflux.

Optionally, effecting and/or conveying comprises having incompetent valves resulting in hydrostatic pressure that prevents upstream venous drainage. Optionally, treating comprises occlusion one or more veins.

In exemplary embodiments of the invention, occlusion comprises one of utilizing a sclerosant, or an element that blocks the vein passage and/or induce thrombosis, or radiation or thermal ablation or ultrasonic or cryogenic ablation, or a combination thereof.

In exemplary embodiments of the invention, a vein comprises at least one of an internal spermatic vein or a deferential vein or a by-pass vein. Optionally, the venous blood is rich in testosterone relative to a normal concentration range in the blood circulation.

An exemplary embodiment of the invention comprises a method for assessing a degree of venous reflux to the prostate comprising:

(a) palpating the prostate for hypertrophy; and

(b) inferring a degree of the reflux responsive to the degree of hypertrophy.

Optionally, the reflux degree is responsive to a unilateral or bilateral reflux or a combination thereof. Optionally, assessing the degree of the reflux comprises assessing a degree of a malfunction of a spermatic vein valves.

An exemplary embodiment of the invention comprises a method for assessing a degree of venous reflux to the prostate comprising:

(a) measuring testosterone concentration; and

(b) determining a degree of the reflux responsive to the testosterone concentration.

Optionally, the testosterone comprises at least one of free or bound testosterone.

In exemplary embodiments of the invention, assessing the degree of the reflux is responsive to the anatomical location of the measurement. Optionally, assessing the degree of the reflux comprises assessing a degree of a malfunction of a spermatic vein valves.

In exemplary embodiments of the invention, the measurement comprises a measurement of at least one of a venous blood or arterial blood. Optionally, the measurement comprises a measurement in a blood vessel at an anatomical location near the prostate before the blood drain to the vena cava.

An exemplary embodiment of the invention comprises a method for reducing aging symptoms comprising effecting a cause that decreased testosterone supply to the blood stream. Optionally, effecting comprises impeding a reflux of venous blood.

In exemplary embodiments of the invention, impeding the reflux comprises treating veins effecting and/or conveying the reflux. Optionally, treating comprises occlusion. Optionally, occlusion comprises utilizing at least one of a sclerosant, an element that blocks the vein passage, an element that induces thrombosis, a material that induces thrombosis, ablation or medication.

In exemplary embodiments of the invention, the vein comprises an internal spermatic vein and/or a deferential vein and/or a by-pass vein.

An exemplary embodiment of the invention comprises a method of passing a wire or a catheter through a venous valve, comprising

(a) expanding a vein such that a valve orifice is opened; and

(b) moving the wire or catheter through the opened orifice.

Optionally, the vein comprises one of an internal spermatic vein, a deferential vein or a by-pass vein.

In exemplary embodiments of the invention, expanding comprises utilizing an expandable element

In exemplary embodiments of the invention, the expandable element is retractable. Optionally or alternatively, the expandable element comprises a wire mesh. Optionally or alternatively, the expandable element comprises an inflatable balloon. Optionally or alternatively, the expandable element comprises an elastic element.

An exemplary embodiment of the invention comprises a guide-wire for passing a catheter through a venous valve comprising:

(a) an expandable element near a distal end of the guide-wire; and

(b) an extension at the distal end of the element, forming a flexible tip.

Optionally, the expandable element is contractible. Optionally, the guide-wire comprises a lumen. Optionally, a control wire passes through the lumen.

In exemplary embodiments of the invention, the control wire effects at least one of expanding or contracting the element. Optionally, pulling the control wire expands the element; and pushing the control wire retracts the element.

In exemplary embodiments of the invention, the expandable element is contractible. Optionally or alternatively, the expandable element comprises a wire mesh. Optionally or alternatively, the expandable element comprises an inflatable balloon. Optionally or alternatively, the expandable element comprises an elastic element. Optionally or alternatively, the expandable element comprises a shape memory material (SMA) expanding or retracting responsive to temperature. Optionally or alternatively, the expandable element comprises a piezoelectric element expanding or retracting responsive to voltage.

An exemplary embodiment of the invention comprises a vascular catheter for occluding a region of a blood vessel comprising:

(a) one or more inflatable elements; and

(b) one or more perforations along at least part of the catheter for a material application.

Optionally, the material is a glue or a sclerosant injected into the catheter or a mechanical element removably attached to the catheter.

In exemplary embodiments of the invention, the length of an interval between the inflatable elements is modifiable.

In exemplary embodiments of the invention, the catheter applies electric field to induce sclerosis.

In exemplary embodiments of the invention, at least some of the perforations are closable.

An exemplary embodiment of the invention comprises a kit for forestalling or therapy, at least partially, of BPH and/or prostate cancer, comprising:

(a) a guide-wire for passing a catheter through a venous valve;

(b) a vascular catheter for occluding a region of a blood vessel; and

(c) an occlusion material or element.

Optionally, the material comprises at least one of a glue, a sclerosant, or an embolization material. Optionally, the element is a coil or an elastic element. Optionally, the element is adapted in size for drainage veins, for example, in a range of about 0.2 mm to 5 mm. Optionally, several different sizes are provided, for example, for allowing treatment of various situations.

In exemplary embodiments of the invention, the kit comprises a plurality of the guide-wire or a plurality of the vascular catheters.

In exemplary embodiments of the invention, the material or elements are sufficient for at least one treatment.

In exemplary embodiments of the invention, the kit comprising an auxiliary device. Optionally, the auxiliary device comprises one of a catheter port, a syringe, or other devices used with the guide-wire or the catheter or the occlusion material or the occlusion element.

An exemplary embodiment of the invention comprises using a sclerosant for the manufacture of medicament for treating BPH or prostate cancer in a subject.

In exemplary embodiments of the invention, the subject age is about 40 or over, 50 or over or 60 or older. Optionally, the subject was not diagnosed as infertile or not diagnosed for varicocele.

### BRIEF DESCRIPTION OF THE DRAWINGS

Non-limiting examples of embodiments of the present invention are described with reference to figures listed below. In the drawings which follow, identical or equivalent structures, elements, or parts that appear in more than one drawing are generally labeled with the same numeral in all the drawings in which they appear. Dimensions of components and features shown in the figures are chosen for convenience and clarity of presentation and are not necessarily shown to scale. Illustrations and labels of the left side of the human male anatomy apply also to the right side, unless specifically indicated.

FIG. 1 schematically illustrates a typical testicular and prostate venous drainage system of a human male;

FIG. 2 schematically illustrates typical testicular and prostate venous drainage paths in a normal left side of a human male;

FIG. 3 schematically illustrates typical testicular and prostate venous drainage paths in a left side of a human male when the one-way valves in the internal spermatic vein do not function.

FIG. 4A schematically illustrates a guide-wire designed to move through venous valves and corners, having an expandable and contractible element in a collapsed state, in accordance with exemplary embodiments of the invention;

FIG. 4B schematically illustrates a guide-wire designed to move through venous valves and corners, having an expandable element in an expanded state, in accordance with exemplary embodiments of the invention;

FIG. 4C schematically illustrates a side view of a distal end of a guide-wire (similar to that of FIGS. 4A and 4B) with expandable and contractible elements, connected to elastic members, in a collapsed state, in accordance with exemplary embodiments of the invention;

FIG. 4D schematically illustrates a side view of a distal end of a guide-wire (similar to that of FIGS. 4A and 4B) with expandable and contractible elements, connected to elastic members, in an expanded state, in accordance with exemplary embodiments of the invention;

FIG. 5A schematically illustrates a catheter for blocking a region and injection within the blocked region, in accordance with exemplary embodiments of the invention;

FIG. 5B schematically illustrates a cross section of the catheter of FIG. 5A perpendicular to the length, in accordance with exemplary embodiments of the invention;

FIG. 5C schematically illustrates a section of the perforated interval between balloons of the catheter of FIG. 5A, in accordance with exemplary embodiments of the invention;

FIG. 5D schematically illustrates a catheter for blocking a region in a vein and injection within the blocked region where the catheter is inside a vein, expanding its walls and blocking the region, in accordance with exemplary embodiments of the invention.

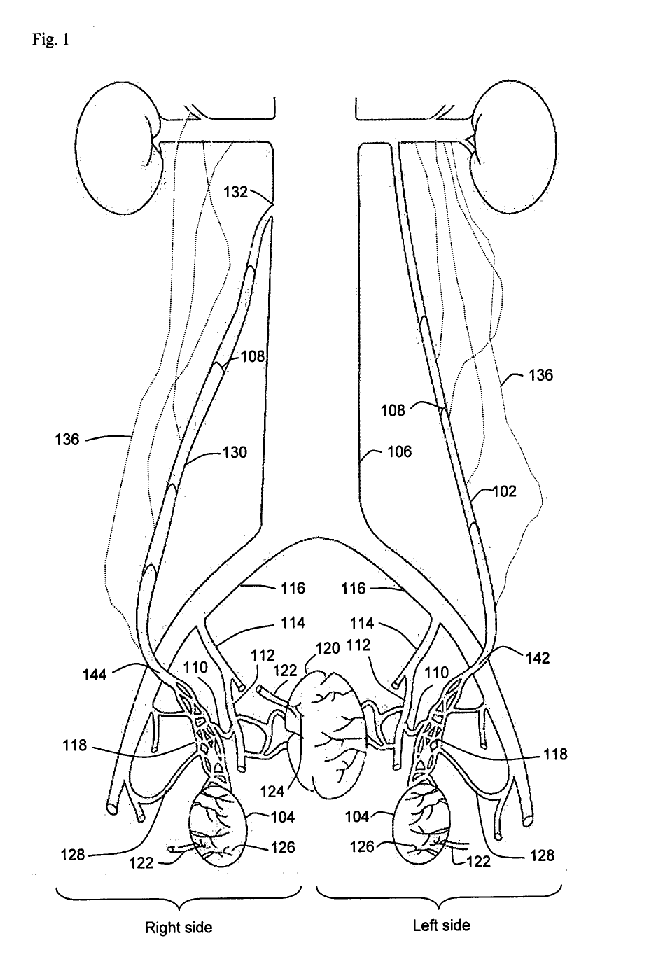

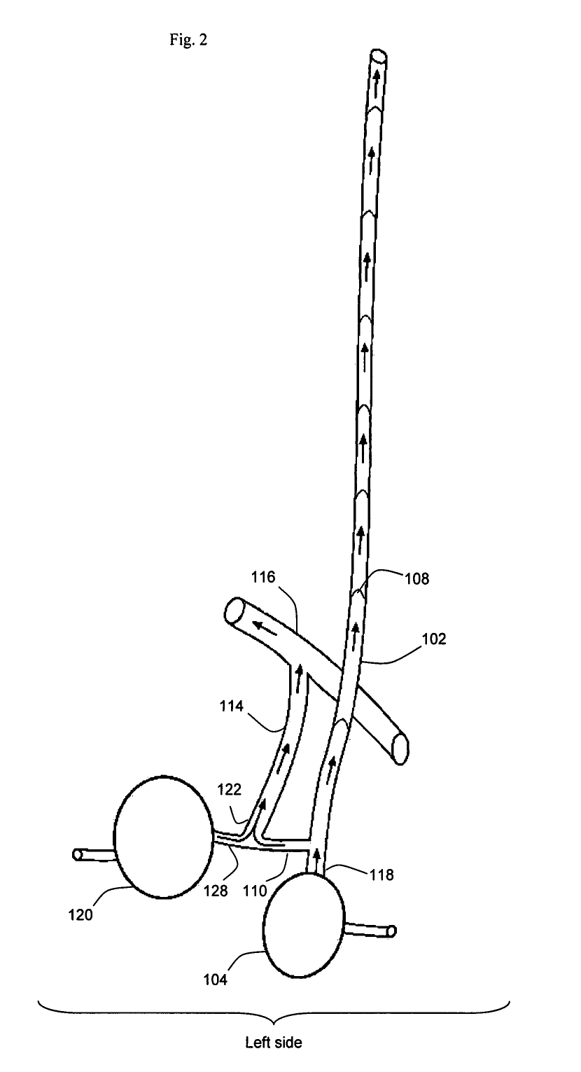

### DETAILED DESCRIPTION OF EMBODIMENTS OF THE INVENTION

In the specifications and claims the terms ‘left side’ and ‘right side’ refer to the conventional anatomical terminology (e.g. the heart, stomach and spleen are on the left side of most human beings). In the specifications and claims the term ‘drainage’ refers to a flow of venous blood via venous vessels towards and into the vena cava, and the terms ‘reflux’ and ‘backflow’ are used synonymously

The headings that follow are not limiting and are intended for clarity only.

### Effects of High Hydrostatic Pressure

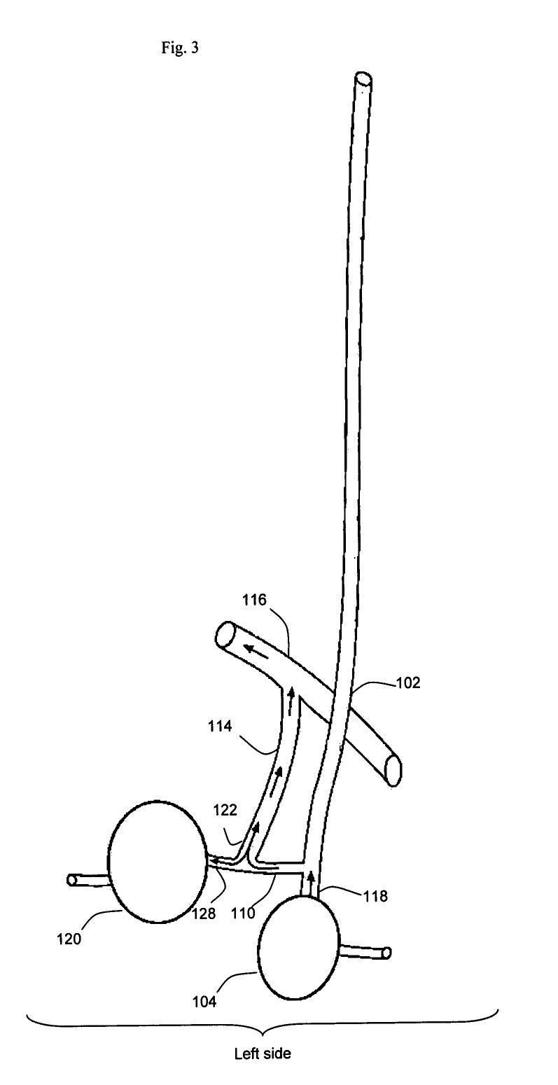

FIG. 3 schematically illustrates typical testicular and prostate venous drainage paths in a left side of a human male when the one-way valves in the internal spermatic vein do not function normally, for example, due to mechanical deterioration such as weakening of valves materials, operational grinding or aging effects.

Since the one-way valves 108 in the internal spermatic vein 102 or 130 do not block back flow (retrograde flow, reflux) down to the testes 104, the internal spermatic veins 102 or 130 form continuous columns of blood in which hydrostatic pressure develops up to approximately 31 mmHg at entry 142 to the left internal spermatic vein 102 approximately 27 mmHg at entry 144 to the right internal spermatic vein 130 (typically about 4-6 fold the typical pressure in ordinary conditions) when the patient is in an upright position such as standing. This excessive hydrostatic pressure, or a pressure of similar magnitude, may exist in vessels connecting to internal spermatic vein 102, such as deferential vein 110 or pampiniform plexus 118, since, according to Bernoulli's law of connecting vessels, the pressure propagates from the testicular to the prostate venous drainage systems and hydrodynamically equilibrates between both drainage systems. The pressure may diminish as vessels are further away from entry 142 or 144, but may be still more than the normal range of about 5 mmHg. The excessive pressure at 142 or 144 and nearby vessels will be denoted as ‘EP’.

This excessive high pressure EP inhibits the drainage of the venous blood from the testes 106 and the pampiniform plexus 118 up the internal spermatic vein 102. Rather, the pressure pushes the testicular venous blood, rich in free testosterone (about 130 fold above serum level), towards the vesicular plexus 128 and onwards to the prostate 120, limiting drainage of venous blood from the prostate.

As the blood still circulates, venous blood from the testis is drained, at least partly, via other paths, such as the deferential vein 110, scrotal vein 128 or by-pass veins 136 that might have developed, possibly due to the excessive pressure.

The excessive pressure EP may produce at least some of the following effects:

(a) The venous blood that is diverted towards prostate 120 and congests and enlarges (dilates) prostate 120. The dilation of prostate 120 may be manifested, at least partially, as BPH or other prostate problems.

(b) The venous blood from the testes 106, rich in free testosterone (relative to a normal level range in the blood circulation), bathes the prostate gland cells with testosterone, effecting benign prostate hyperplasia (BPH). About 90% of the free testosterone is irreversibly converted to dehydrotestosterone (DHT), which has about five fold higher affinity for androgen receptors than free testosterone and may effects an accelerated proliferation of prostate cells. It should be noted that due to the short passage from testes 104 to prostate 120 (about 10-15 cm), only a small amount of free testosterone is bound to SHBG or albumin before bathing the prostate receptors.

(c) The excessive pressure EP and congestion of the prostate inhibits or reduces arterial blood from entering microcirculation 124 of the prostate and disrupts the biological balance. The excessive amounts of testosterone and DTH present in the prostate may induce an accelerated proliferation of prostate cells, and promote the development of cancer. It should be noted that the extreme concentration of free testosterone (about or over 100 fold relative to normal) in the prostate may overload the DNA hormonal feed back system, and increase the probability of mutations in the accelerated cells divisions.

(d) The excessive venous pressure EP inhibits or reduces arterial blood from entering the microcirculation 126 of the testes. The blood stagnates to at least some extent, and oxygenated arteriolar blood cannot flow normally into the testis, resulting in degenerative processes in the testes tissues which diminish its testosterone production.

(e) The impaired testosterone production, resulting in reduced testosterone in the blood serum, may effect aging expressions or symptoms.

### Remedy of High Hydrostatic Pressure and Some of its Effects

In exemplary embodiments of the invention, one or more of the adverse states and effects described above may be avoided, delayed, alleviated and/or repaired, at least to some degree, by reducing or eliminating the excessive pressure EP. Reducing the pressure reduces or eliminates the back flow (reflux) of venous blood, rich in testosterone (relative to normal levels in the blood circulation), from the testes to the prostate.

In exemplary embodiments of the invention, the reflux is prevented or impeded by occlusion (e.g. embolization or sclerosis), of the left internal spermatic vein 102 and/or the right internal spermatic vein 130 that has effected the excessive hydrostatic pressures EP. Optionally and additionally, some or all veins through which the reflux flows, such as the deferential vein 110 and the pampiniform plexus 118, are occluded. Optionally or additionally, bypass veins 136 that might have developed are occluded too.

In exemplary embodiments of the invention, deferential vein 110 is occluded to block the backflow of testicular venous blood into the prostate, relieving it of the excessive pressure and high testosterone, and allowing it to recover. Optionally, other veins are not treated for backflow and/or varicocele, at least for a certain time duration.

In exemplary embodiments of the invention, occluding only deferential vein 110 may be beneficial in case of metastases or suspicion for metastases, since testosterone drainage to the bloodstream via the internal spermatic veins 102/130 is inhibited by the excessive pressure EP, and blocking also the passage via deferential vein 110 results in reduced testosterone supply to the bloodstream, and possibly reduced risk for metastases proliferation. Optionally, deferential vein is occluded using microsurgery, and optionally be exposing the vein. Optionally, the surgery is conducted under ultrasound or other imaging guidance. Optionally, other veins are treated during the operation.

Once the excessive pressure EP is reduced, venous blood from the testes may use alternative paths to drain to the inferior vena cava, such as through a scrotal vein 128. Arterial blood may now enter the testes microcirculation unimpeded 126, and restoring, at least partially, testosterone production and allow recovery of damaged tissues.

Furthermore, the prostate is relieved of the back flow and swelling, and it can drain the excessive blood congestion with testosterone via the vesicular plexus 128. With the excessive venous pressure relieved, arterial blood can more easily enter the prostate microcirculation 124. Optionally or additionally, the recovering prostatic tissue, with arterial blood with approximately normal testosterone levels (and bound serum testosterone) could reduce the stimulus to growth of cancer tissues in the prostate. Optionally and additionally, anti-androgenic agent may be administered, locally or systemic, to further the healing effect. Optionally, the additional medication may lower even more the testosterone levels without affecting the patient health.

Restoring, at least to a certain extent, the testosterone levels in the blood may reduce symptoms of aging or other effects that were induced due to testosterone deficiency.

In exemplary embodiments of the invention, the occlusion is carried out by applying sclerosants (sclerosing agents) into a vein. The sclerosant may be, for example, Sodium tetradecyl sulfate (Sutra-Decol), alcohol or its derivatives, Cyanoacrylate, N-butyl-2-cyanoacrylate (NBCA) (‘glue’), Onyx, PVA particles, acrylic microspheres or any blocking agent of the art. Optionally, the sclerosant is applied via intravenous catheter or catheters. Optionally or alternatively, the sclerosant is applied subcutaneously, such as by a syringe. Optionally, other methods of blood vessels blocking are used, such as placement of coils, or other elements such as silk (optionally coated with sclerosant or other materials) that block the vein lumen and/or induce thrombosis that blocks the vein and typically induces degeneration and permanent occlusion. Optionally, endovascular ablation such as radiofrequency radiation that heats up the vein, or application of direct heating, is used to damage the vein and/or induce its walls to shrink and/or develop a thrombosis, optionally a complete blocking of the vessel. Optionally, a friction with the vessel endothelium may be used to shrink and occlude the vessel. Optionally, electrocautery such as by electric wire in a catheter, or laser heating by an optic fiber in the catheter may be used to heat and shrink the vessel or effect sclerosis. Optionally, these methods are applied by minimally invasive methods such as by laparoscopy. Optionally, the methods are applied externally such as by or radiation, for example, a plurality of laser beams is used to focus at the sclerosis region, while each beam does not damage, or negligibly damage, the other tissues whereas the convergent beams at the focus have sufficient power to shrink and/or effect sclerosis of the vein. Similarly, electromagnetic radiation (e.g. x-ray or by MRI) from several directions focusing at the sclerosis region may be used. Similarly, ultrasound radiation or cryogenic methods may be used to shrink the blood vessel. Optionally, other mechanical, biological, chemical or physical methods and/or mechanisms, or a combination of said methods and mechanism, may be used to block the blood vessel. Optionally or additionally, a temporary embolization such as by Gelfoam (gelatin powder) which clots the vessel and later on dissolves may be used, at least partially.

In exemplary embodiments of the invention, a sclerosant is used for the manufacture of a medicament for forestalling and/or treating BPH or prostate cancer in a subject. Optionally, the sclerosant is adapted to treating backflow that effects BPH and/or prostate cancer. Optionally, the adaptation comprises the composition of materials and/or their proportions, for example, mixing two or more occlusion materials, optionally comprising temporary occlusion material such as Gelfoam. Optionally and additionally, the medicament may comprise materials with affinity to testosterone and/or adapted to bind to and occlude vessels containing high concentration of bound and/or free testosterone. Optionally, the high concentration comprises 5 to 10 fold, or higher (e.g. 50 to 100 fold), than the normal range of bound and/or free testosterone. Optionally, the medicament is administered systemically or locally.

In exemplary embodiments of the invention, an antiandrogen medication such as an antigen bound guided molecular therapy may used as part of the treatment. Optionally, the antigen reduces testosterone production by affecting regions in the brain (e.g. hypophyse or hypothalamus) that regulate testosterone production. Optionally, the antiandrogen comprises materials such as is LHRH analogs (luteinizing hormone-releasing hormone), administered systemically or as subcutaneous patch. Optionally, such antiandrogen material may be a part of the medicament described above.

In exemplary embodiments of the invention, the occlusion treatments are useful in forestalling prostate cancer metastases by either (a) occlusion as described above, preventing the development of cancer, and hence, metastases, or (b) if cancer is already present, occlusion (e.g. by microsurgery) of at least the deferential vein or other vessels that drain from the prostate to the blood stream. The occlusion blocks at least some of the venous passage from the prostate and consequently reduces possible leakage of cancerous cells from the prostate that my settle at certain organs and additionally, may reduce testosterone supply to the bloodstream, reducing the risk of metastases proliferation.

In exemplary embodiments of the invention, deferential vein 110 may be reached by a catheter or other methods via the femoral vein to the common iliac vein to internal iliac vein to internal spermatic vein and to the deferential vein. Optionally, the path is from the vena cava to the renal vein to the internal spermatic vein and to the deferential vein. Optionally, an antegrade approach may be used. Optionally or alternatively, the deferential vein may by separated and exposed, allowing direct treatment thereof.

### Varicocele/Reflux Diagnosis

The deterioration of one-way valves 108 (typically apparent as varicocele), that result in excessive high hydrostatic pressure EP, can cause the prostate to enlarge (hyperplasia) as discussed above.

In exemplary embodiments of the invention, the level of varicocele, or the degree of valve degradation or malfunction, or the degree of the clinical significance of the valves degradation in a spermatic vein and/or the degree of venous blood reflux to the prostate, may be assessed or diagnosed responsive to the degree of prostate hyperplasia.

In exemplary embodiments of the invention, prostatic hyperplasia may be diagnosed or assessed by palpation or other methods such as diagnostic imaging. Optionally, the diagnosis is responsive to a unilateral (left or right side) malfunction of valves 108 or bilateral (left and right side) malfunction, or a combination of the malfunction levels of each side. Optionally, the malfunctioning side may be identified by methods such as radiology or ultrasonography.

In exemplary embodiments of the invention, the degree of varicocele, and/or the degree of valve degradation in the spermatic veins and/or the degree of venous blood reflux to the prostate, may be assessed or diagnosed based on the testosterone concentration measured near a testes or the prostate or between them. Optionally, the serum testosterone is measured at the lower part of the internal spermatic vein, or at or above pampiniform plexus 118, or the deferential vein 110, or vesicular plexus 128. Optionally, the testosterone level is measured using a syringe or a catheter or by non-invasive methods such as radiation (e.g. laser or infrared). Optionally, varicocele is diagnosed visually or by palpation, and if present to a certain extent, which may indicate a potential increased risk of cancer, testosterone concentration is consequently measured. Optionally, free testosterone concentration is measured for varicocele diagnosis. Optionally, total testosterone is measured. The relation between the varicocele and testosterone concentration is based on the location where the measurement was made. For example, the measurement at the vesicular plexus 128 may be more indicative or credible than at a pampiniform plexus 118 because elevated testosterone concentration at the vesicular plexus is due to reflux whereas high concentration at the pampiniform plexus may be, at least partially, due to testosterone that did not yet enter the systemic circulation. Optionally, the measurement is repeated, or a plurality of measurements are taken at a plurality of anatomical locations. Consequently, an average, or other estimation or statistical derivation, may yield a more stable and/or reliable relation. Optionally, testosterone is tested at the blood circulation so that a difference may be assessed for diagnosis. Optionally, a range of normal and pathological concentrations are compiled for comparison and diagnosis.

### Guide-Wire

In order to access the internal spermatic vein 102 or 130 and below, such as to the pampiniform plexus 118 or the deferential vein 110, a catheter should pass the one-way valves 108 which normally resist flow against the normal flow direction. Since some of the valves may still be functional in patients with this condition, at least partially, it takes a special skill and training to perform the maneuver without damaging the valves.

Furthermore, the right internal spermatic vein 130 frequently enters the inferior vena cava 106 at an angle 132, such that a catheter being advanced from below (e.g. from the femoral vein) needs to take a sharp turn while accessing a valve or valves 108.

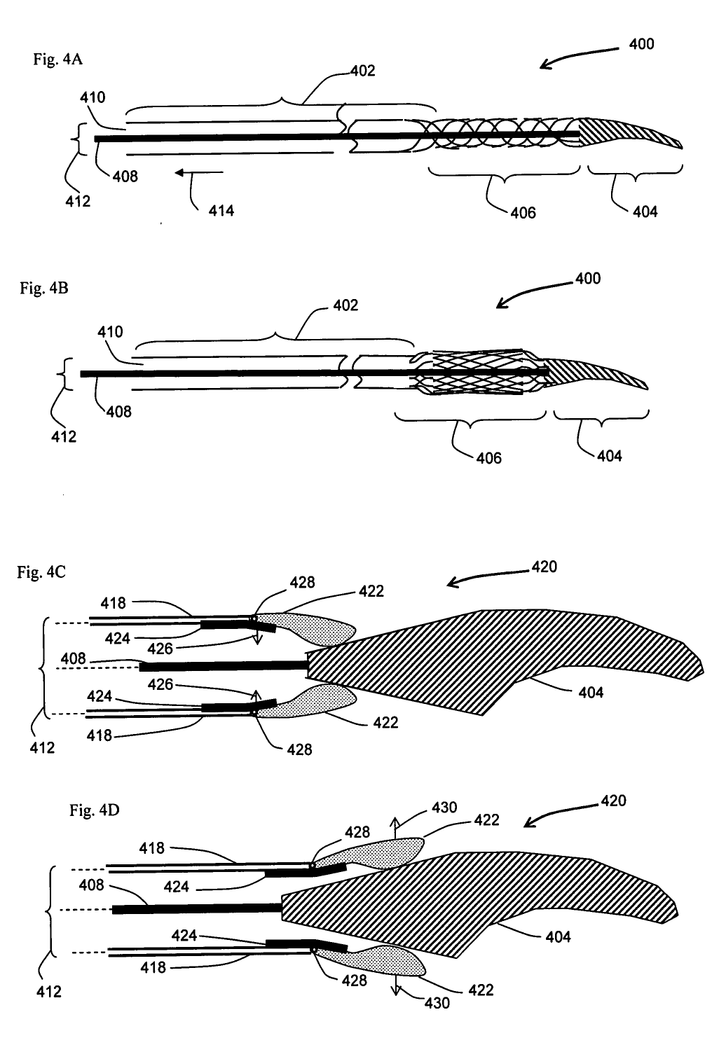

FIG. 4A schematically illustrates a guide-wire 400 designed to move through venous valves and junctions such as 132, having an expandable and contractible element 406 in a collapsed state, while FIG. 4B schematically illustrates the guide-wire 400 having an expandable element 406 in an expanded state, in accordance with exemplary embodiments of the invention.

In exemplary embodiments of the invention, the guide-wire 400 comprises:

(a) an expandable and contractible element 406 near the distal end of guide-wire 400; and

(b) an extension 404 at the distal end of element 406, forming a flexible tip 404;

In exemplary embodiments of the invention, guide wire 400 comprises an elongated duct 402 having a lumen 410. Optionally, a guide wire 400 comprises a control wire 408 that passes through lumen 410. Optionally, control wire 408 passes through the expandable element 406 for expanding and contracting element 406.

In exemplary embodiments of the invention, an operation of the guide-wire comprises:

(a) manipulating tip 404 to maneuver guide-wire 400 to reach a vein near a valve 108 (sufficiently close for the subsequent operation below);

(b) pulling control wire 408 from the proximal end of guide-wire 400 in direction 414 thereby pushing distal end 404 against element 406 and compelling element 406 to expand against the vein walls. As element 406 expands, it stretches and widens the vein, thereby opening the orifice of the nearby valve; and

(c) pushing control wire 408, collapsing back element 406, while passing guide-wire 400 through the valve orifice before it constricts back.

In exemplary embodiments of the invention, an intravascular catheter (not shown) is maneuvered to the proximity of a valve such as 108, and the operator injects a contrast agent via the catheter to visualize the valve and the position of the catheter. Once the operator is satisfied that the intended position is reached, the guide-wire is inserted in the catheter and manipulated, aided by the tip 404, to reach near a valve 108. The element is expanded, opening the vessel walls and the valve orifice. The control wire is operated as describe above. Once the wire has passed the valve, a catheter can be pushed through the open valve, optionally over guide wire 400.

In exemplary embodiments of the invention, guide wire 400 with flexible tip 404 allows easy maneuvering in the blood vessels and element tip allows fast passage through venous valves, for easy insertion of catheters into the vein.

In exemplary embodiments of the invention, the element 406 comprises a collapsible wire mesh. Optionally, the mesh is twisted in the collapsed state and untwisted in the expanded state. Optionally, by pulling the control wire 408 the mesh is pressed against distal end of duct 402 and compelled to expand. Optionally the mesh expansion is by unwinding the spiraled grid wires 408 as the mesh is pulled by control wire 408. Optionally, the collapsed mesh is elongated relative to the expanded state.

In exemplary embodiments of the invention, the expansion of element 406 by control wire 408 is carried out about 2-3 cm upstream of a valve 108. Optionally, the distance is different, optionally to fit the expansion of the vein and valve orifice.

In exemplary embodiments of the invention, the expansion is approximately by 5 mm diameter. Optionally the expansion is by 4-6 mm. Optionally it is more than 6 mm. Optionally, the expansion is less than 4 mm. Optionally, the expansion is according to the vessel diameter. Optionally, the expansion is adjustable for the intended vessel or valve, e.g. by twisting or untwisting the mesh before use.

In exemplary embodiments of the invention, element 406 width in its collapsed state is approximately the same as guide-wire 400 width 412. Optionally, width 412 is approximately 0.018 inch. Optionally width 412 is adapted to the operation or the vessels diameter. Optionally, width 412 is adjustable e.g. by twisting or untwisting duct 402.

In exemplary embodiments of the invention, the mesh may be replaced, at least partially, by other mechanisms. For example, an inflatable balloon, or other extendable/contractible mechanism such as by elastic elements or elements which, optionally, operated by the control wire, for example, as describe with respect to FIG. 4C below.

FIG. 4C schematically illustrates a side view of a distal end of a guide-wire 420 (similar to guide-wire 400 of FIG. 4A and 4B) with expandable and contractible elements 422, connected to elastic members 426, in a collapsed state, and FIG. 4C schematically illustrates the side view in an expanded state, in accordance with exemplary embodiments of the invention.

In exemplary embodiments of the invention, element 422 comprises a plurality of elements around the distal end of walls 428 of duct 402. Elements 422 are rotatable about pivot 428 on walls 418 of duct 402 and connected to elastic elements 426 which normally push inwards into lumen 410 in directions 426.

Elements 422 touch tip 404 firmly due to the pressure force of elastic elements 426. Elements 422 and tip 404 are shaped such that when control wire 408 is pulled towards the proximal end of guide-wire 420, tip 404 moves towards lumen 410, while forcing and pushing elements 422 outwards in directions 430 against the pressure of elastic elements 426. Expanded elements 422 push against a vein's wall, stretching and widening the wall and compelling the orifice of a nearby valve 108 to open. Pushing control wire 408 towards the distal end, control wire 108 pushes tip 404 while elements 422 contract under the pressure force of elastic elements 426 in directions 426, letting guide-wire 420 pass through the open orifice.

In exemplary embodiments of the invention, other mechanisms may be used to expand the walls of a vein, for example, a piezoelectric element that expands by voltage passed by a wire in the catheter, or an element comprising a shape memory alloy (SMA) expanding and/or contracting responsive to temperature, for example, by injections into lumen 410 a liquid, such as saline or plasma, at different temperatures

In exemplary embodiments of the invention, tip 404 is flexible and manipulated by the distal end of guide wire 400 to enter left internal spermatic vein, optionally beginning with the femoral vein or otherwise. Optionally, tip 404 is manipulated to enter the right spermatic vein 132 via corner 108.

In exemplary embodiments of the invention, the guide-wire is used for valves in the internal spermatic veins. Optionally and alternatively, valves 108 are near a junction where veins join such as that of the left renal vein and left spermatic vein, or the inferior vena cava and the right spermatic vein (132). These locations are typically difficult to enter due to the sharp angle of the bending. Optionally and additionally guide-wire 400 is suitable for entering and passing through a sharply angled vascular turn.

### Occlusion Catheter

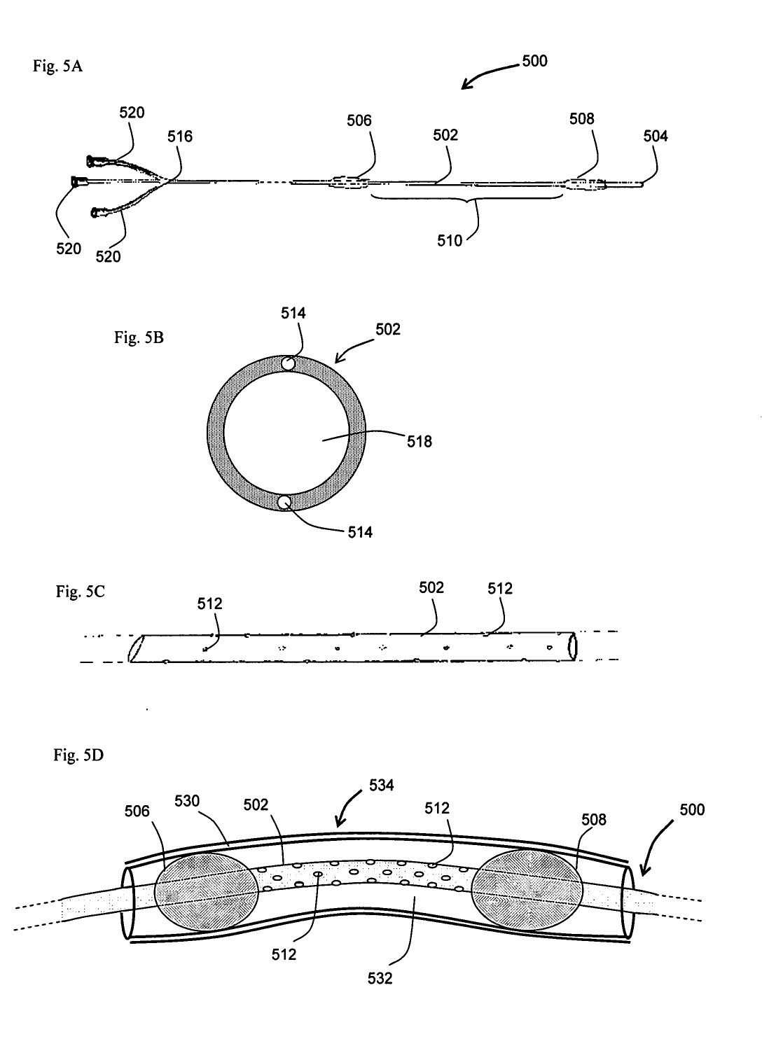

FIG. 5A schematically illustrates a catheter 500 for blocking a region and injection within the blocked region, and FIG. 5B schematically illustrates a cross section of the catheter of FIG. 5A perpendicular to its length, in accordance with exemplary embodiments of the invention.

In exemplary embodiments of the invention, the catheter is intended for occluding a volume in a vascular region and injecting material into that region limiting drainage of the injected material out of the region. Optionally, the region comprises a venous intersection (e.g. 132).

In exemplary embodiments of the invention, the catheter comprises:

(a) an elongated tube 502 having a lumen 518;

(b) two inflatable elements 506 and 508 separated by an interval 510, one element 508 near the distal end of the catheter; and

(c) one or more perforations 512 along interval 510 between elements 506 and 508, as shown in FIG. 5C.

In exemplary embodiments of the invention, the inflatable elements 506 and 508 comprise balloons. Optionally, the catheter 500 comprises more than two balloons. Optionally, the catheter comprises one balloon. Optionally or additionally, a balloon inflates at its two ends or at a plurality of locations along its length and may occlude (isolate) a region between the inflated locations. Optionally, the number and structure of the balloons are adapted to prevent flow out of intended region 532.

In exemplary embodiments of the invention, catheter 500 comprises one or more lumens 514 between proximal 516 end of catheter 500 and the balloons, as shown, for example, in FIG. 5B.

Optionally, catheter 500 comprises one or more ports 520 at the proximal end of the catheter. Optionally, a port 520 connects to lumen 518, and two ports 520 connect to each tubes 514 leading to a balloon. Optionally, one port 520 connects to both tubes 514. Optionally, ports 520 are used to inflate and deflate the balloons and/or to inject material into the vein and/or to aspirate the vein.

Optionally, at least some of the perforations 512 are closable, for example by valves operated by a mechanism such as a control wire, or by insertion of a tube inside catheter 500 to cover some of the perforations (without disrupting the catheter mechanism and operation).

In exemplary embodiments of the invention, an operation of the catheter comprises:

(a) maneuvering the catheter so that the interval 510 is about the intended region for injection;

(b) distal balloon 508 is inflated via a port 520 and a tube 514 connected to the balloon, until the balloon contacts the vessel walls with sufficient force to prevent drainage of blood and/or injected material.

(c) proximal 506 balloon is inflated via a port 520 and a tube 514 connected to the balloon, forming a closed region about the interval;

(d) a material is injected (or installed) from port 520 via the catheter lumen 518 and out of the perforations 512;

(e) deflating the balloon and removing the catheter from the region.

Optionally, step (c) may precede step (b).

In exemplary embodiments of the invention, removing the catheter in step (e), takes into account the nature of the material. For example, if it is a liquid then the catheter is pulled while the material is wet, or if it is a mechanism or elements, it is pulled without detaching the mechanisms or elements.

In exemplary embodiments of the invention, one balloon may be inflated to block flow of material to an undesirable location while letting the material to flow towards the opposite direction.

Optionally, the blood from the vein about the interval 510 is aspirated via the perforations 512 before injection. Optionally and alternatively, a suction is used to collapse the vein.

FIG. 5D schematically illustrates catheter 500 for blocking a region 532 in a vein 534. Balloons 506 and 508 are inflated, expanding vein 534 walls 530 and blocking region 532, in accordance with exemplary embodiments of the invention.

In exemplary embodiments of the invention, the inflatable balloons are replaced, at least partially, by other blocking elements. For example, a removable coil or an elastic element or other elements. Optionally, perforations 512 are adapted in size and/or shape to install the blocking elements.

In exemplary embodiments of the invention, either instead or with the inflatable balloons (506 and/or 508) a welding element is opened towards the walls of a vessel and welds and occludes the vessel lumen. Optionally, the welding comprises electrically heating.

In exemplary embodiments of the invention, the material comprises mechanical elements such as a coil or other elements.

In exemplary embodiments of the invention, the catheter is intended, but not limited to, for sclerosis of an internal spermatic vein 102 or 130.

In exemplary embodiments of the invention, distal 504 end is either open or closed. Optionally, a closed distal end 504 is openable (e.g. by inflating a balloon), for example to allow a passage of a guide-wire or another catheter, or for injection or aspiration at the end.

In exemplary embodiments of the invention, the length of interval 510 is designed to fit certain anatomies or venous regions. For example, a deferential vein 108 is shorter than an internal spermatic vein so that a shorter interval 510 may be required. Optionally or alternatively, interval 510 is modifiable, for example, by having a plurality of balloons that inflate according to the required interval, or having a telescopic structure operated by a control cord or a guide-wire in the catheter lumen. Optionally, the length of interval 510 is such as to contain sufficient amount of sclerosant to ensure proper occlusion, or to allow the deployment of mechanical elements such as coils.

In exemplary embodiments of the invention, the catheter uses the guide-wire 400 for moving through venous valves.

### Exemplary Kit

An exemplary embodiment of the invention comprises a kit for use in forestalling and/or treatment of BPH and/or prostate cancer. Optionally, the kit comprises:

(a) A guide-wire as described above;

(b) A vascular catheter as described above; and

(c) an amount of occlusion material or element.

Optionally, the occlusion material comprises a glue or a sclerosant or an embolization material. Optionally, the element is a coil or an elastic element.

Optionally, the kit comprises a selection of a glue and/or sclerosant and/or embolization material, Optionally, the material or elements are sufficient for one or for more than one typical treatments, for example, amounts suitable for treating two sides, in 80% to 95% of the cases.

Optionally, the kit comprises a plurality of guide-wires and/or catheters.

Optionally, the kit comprises auxiliary devices such as one or more catheter ports for injection or aspiration, and/or one or more syringes for injecting material to the catheter. Optionally, the kit comprises other devices that are typically required or used with the guide-wire or the catheter or the occlusion material or the occlusion element.

Optionally, the kit includes instructions for use, for example in writing and/or drawings.

### Exemplary Patients Screening Procedure

Exemplary screening patients for treatment comprises one or more of the following procedures, or a combination thereof.

I: Selecting patients complaining of nocturia, at least for some time. Priority is given to age and/or frequency and/or urgency of urination. Alternatively, diagnosis or assessment for hypertrophy of the prostate is performed by palpation of the prostate.

Alternatively or additionally, thermography may be used for non-invasive diagnosis of prostate disease which may be related to varicocele.

Optionally or additionally, the diagnosis or assessment of a malfunction or disorder of the prostate comprises measuring testosterone levels near the prostate.

In exemplary embodiments of the invention, the diagnosis results are compared and/or combined to achieve a more reliable judgment relative to one method. Optionally, the diagnosis is repeated at least twice to avoid uncertainties.

II: Measuring prostate size, for example, by ultrasound and/or palpation. Patients with significantly large prostate size and/or abnormal clinical state are selected, wherein a normal range is about 15-20 ml, depending also on age.

Optionally and additionally, thermographs may be used to assess directly elevation of the temperature in the ISV indicating that venous backflow exists, suggesting that there is elevation of hydrostatic pressure in the testicular-prostatic drainage systems (e.g. by a contact thermography using a flexible liquid crystal thermostrip (FertiPro, Breemen, Belgium)). Optionally, the thermographs are taken at a testis wherein an elevated temperature (above a typically about 32° C., e.g. about 37° C.) indicates a backflow of systemic blood into the testis.

III: Selecting patients over a certain age such as about 40 or over 50 or over 60 or older.

IV: Selecting patients without fertility problems.

V: Selecting patients with prostate problems with no apparent and/or palpatable varicocele.

VI: Selecting patients having right side varicocele or impaired right side venous drainage.

VII: Selecting patients having left side varicocele or impaired left side venous drainage.

VIII: Selecting patients known to have prostate cancer or BPH.

IX: Selecting patients with complaints or clinical symptoms judged to be related to prostate.

X: Selecting patients with complaints or clinical symptoms judged to be related to varicocele.

The order of the procedures, and/or alternatives, are at the discretion of the physician.

Screening for cancer may be achieved, for example, by biopsy of the prostate.

Exemplary Treatment Procedures for Prostate BPH and/or Cancer

I: Occlusion treatments as described above in the specifications, and/or in the occlusion procedures outlined below.

II: Augmenting, or combining, the occlusion treatment with additional therapy to ensure that BPH and/or cancer are cured satisfactorily. For example, occlusion may be combined with ablation (e.g. thermal, radiation, ultrasound, cryogenic) of veins that allow backflow to the prostate, such as the deferential vein. The combination may serve as a preventive measure to forestall the development of prostate hypertrophy to BPH, or to forestall the development of BPH into cancer. Another example is when prostate cancer is present and occlusion of the deferential vein, and/or other veins which allow backflow, is augmented with chemotherapy. Chemotherapy may be administered systemically (e.g. by transfusion) or locally at the prostate or its surroundings (e.g. by a catheter or subcutaneously). The augmentation by chemotherapy may also prevent and/or treat metastases. Optionally, radiation therapy (e.g. brachitherapy or external radiation from a Linac or gamma radiation) may be used to augment the treatment of cancer.

III: Augmenting, or combining, the occlusion with anti-androgenic agent administration, optionally with reduced dosage relative to conventional androgenic treatments.

In exemplary embodiments of the invention, typical quantities of sclerosant comprise a range of about 0.2 cc to 10 cc. For example, for the left spermatic vein about 5 cc may be used, and for the right spermatic vein about 4 cc may be used, and for the deferential vain about 0.5 cc to 1 cc may be used.

### Exemplary Occlusion Procedures

### Femoral Vein Access

A “One stick” trocar type venous access sheath: 6 French (2 mm) inner diameter flushable sheath placed with ultrasound guidance for rapid access.

### Catheter Navigation in Inferior Vena Cava and Left Renal Vein

1) Using a left internal spermatic vein catheter with tip angled 20 degrees relative to the anterior plane of catheter to facilitate rapid access to left internal spermatic vein when it joins the left renal vein along its anterior wall.
2) Using a left internal spermatic vein catheter with moveable tip where the distal 1 cm can be flexed or extended by 20 degrees to facilitate entry into the left internal spermatic vein by pressing on side of orifice of left internal spermatic vein to allow opening of closed valve.
3) Use of fluoroscopic table tilting in Trendelenburg position where the head is down about 20-30 degrees (e.g. to reduce hydrostatic pressure) during attempts to pass through competent valves.
4) Using a fluoroscopic table tilting in reverse Trendelenburg position (head up about 30-90 degrees) to facilitate and maximize imaging of internal spermatic vein reflux.

### Vessel Occlusion

1) Use of manual compression over inguinal canal for the diagnostic study of the anatomy of the internal spermatic vein prior to injection of sclerosing agent to ensure that there is no reflux of the subsequent sclerosing agent into the pampiniform plexus during sclerotherapy.
2) Use of absolute alcohol to occlude internal spermatic vein with double balloon occlusion catheter.
3) Using of hot iodinated contrast material to occlude internal spermatic vein.
4) Alternatively or additionally, using 50% glucose to occlude internal spermatic vein.
5) Use of low voltage (1-3 volt) direct current electrified guidewire to induce venous spasm prior to occlusion by sclerotherapy.
6) Use of cyanoacrylate adhesives in conjunction with sclerosing agents to achieve thorough occlusion of main ISV and its tributaries and potential collateral vessels.
7) Use of other sclerosing agents, including Ethanolamine, Polidecanol to facilitate occlusion.
8) Use of catheter to identify, isolate and occlude the connection between the internal spermatic vein and the deferential vein for the purpose of treating prostatic disease.

### Right Internal Spermatic Vein Access

1) Reversed curve guiding catheter with flared, blunted tip to sit in orifice of internal spermatic vein. Aspiration through the catheter should allow opening of a competent orifice valve.
2) Optionally using vasodilatory agents such as sublingual nitrates, Cordil, or Viagra to induce venous dilatation and facilitate entry into the internal spermatic vein.
3) Use of intravenous anesthesia incorporated into sclerosing agent to prevent venous spasm during treatment and induce anesthesia.

### Exemplary Non-Catheter Applications

Direct access of the deferential vein using ultrasound guidance to allow selective percutaneous occlusion of the connection between the internal spermatic vein and the prostatic venous plexus.
1) Direct access to the internal spermatic vein at the base of the scrotum to allow visualization of otherwise occult collateral veins which have caused recurrence of varicocele in previously treated cases.
2) Direct transrectal puncture of the prostatic venous plexus to permit antegrade visualization of potential anastomoses with the internal spermatic vein by injecting intravenous contrast and fluoroscopic radiological imaging. This is to allow recognition of potential pathways for metastatic spread in cases of prostatic cancer before percutaneous sclerotherapy of the internal spermatic vein.
3) Optionally or alternatively, using microsurgery and/or subcutaneously injection of occlusion materials.

### Exemplary Studies

### Exemplary Study 1

Before treatment all 9 patients (in ages 36-67) reported nocturia (ranging from one to eight times a night), with a prostate volume (measured by ultrasonography) in the range of 21-52 ml. Three to six weeks after occlusion treatment the prostate volume decreased to 12-30 ml and nocturia decreased to a 0-2 time a night.

### Exemplary Study 2

A 69 years old patient was diagnosed (November 2005) with prostate cancer after a PSA screening test (14.5 ng/ml). Digital rectal examination revealed an enlarged prostate but otherwise unremarkable physical examination with no suspicious nodules. On trans-rectal ultrasound (TRUS) a hypoechogenic region was found (11 mm) in his right lobe. Two of ten biopsy scores both from the right side revealed adenocarcinoma of the prostate with a Gleason score of 3+3=6 in about 5% of the tissue. The patient was treated with one injection of goserelin (December 2005) and then referred to an oncologist. After a long discussion on all possible treatment options for low risk prostate cancer the oncologist opted for an active surveillance. On April 2006 the patient underwent occlusion of bilateral ISV including the whole network of associated bypasses (using x-ray imaging for guidance) and sclerotherapy (embolization) of bilateral varicocele, which was diagnosed after complaints of testicular discomfort. Despite an elevation of blood testosterone levels to the normal range (from May 2006), PSA dropped markedly and remained steady at 2 ng/dl (last test on January 2007). Repeated TRUS demonstrated a continuous decrease in the prostate size (41.4 cc at diagnosis down to 27.4 cc on December 2006) and the suspicious hypoechogenic area in his right lobe (seen by TRUS) disappeared. Repeat biopsies of the right lobe (December 2006) showed no evidence of cancer.

### General

In the description and claims of the present application, each of the verbs “comprise”, “include” and “have” as well as any conjugates thereof, are used to indicate that the object or objects of the verb are not necessarily a complete listing of members, components, elements or parts of the subject or subjects of the verb.

The present invention has been described using detailed descriptions of embodiments thereof that are provided by way of example and are not intended to necessarily limit the scope of the invention. In particular, numerical values may be higher or lower than ranges of numbers set forth above and still be within the scope of the invention. The described embodiments comprise different features, not all of which are required in all embodiments of the invention. Some embodiments of the invention utilize only some of the features or possible combinations of the features. Alternatively and additionally, portions of the invention described/depicted as a single unit may reside in two or more separate physical entities which act in concert to perform the described/depicted function. Alternatively and additionally, portions of the invention described/depicted as two or more separate physical entities may be integrated into a single physical entity to perform the described/depicted function. Variations of embodiments of the present invention that are described and embodiments of the present invention comprising different combinations of features noted in the described embodiments can be combined in all possible combinations including, but not limited to use of features described in the context of one embodiment in the context of any other embodiment. The scope of the invention is limited only by the following claims.

All publications and/or patents and/or product descriptions cited in this document are fully incorporated herein by reference to the same extent as if each had been individually incorporated herein by reference or if they were reproduced in full herein.

1. A method for forestalling or therapy, at least partially, of BPH or prostate cancer comprising:
(a) identifying a reflux of venous blood to the prostate; and
(b) impeding the reflux by treating veins effecting or conveying the reflux.

2. A method according to claim 1, wherein effecting or conveying comprises having incompetent valves resulting in hydrostatic pressure that prevents upstream venous drainage.

3. A method according to claim 1, wherein treating comprises occlusion one or more veins.

4. A method according to claim 3, wherein occlusion comprises utilizing a sclerosant.

5. A method according to claim 3, wherein occlusion comprises utilizing an element that blocks the vein passage or induce thrombosis.

6. A method according to claim 3, wherein occlusion comprises radiation or thermal or ultrasonic or cryogenic ablation, or a combination thereof.

7. A method according to claim 1, wherein treating comprises a combination of methods.

8. A method according to claim 1, wherein a vein comprises at least one of an internal spermatic vein or a deferential vein or a by-pass vein.

9. A method according to claim 1, wherein the venous blood is rich in testosterone relative to a normal concentration range in the blood circulation.

10. A method for assessing a degree of venous reflux to the prostate comprising:
(a) palpating the prostate for hypertrophy; and
(b) inferring a degree of the reflux responsive to the degree of hypertrophy.

11. A method according to claim 10, wherein the reflux degree is responsive to a unilateral or bilateral reflux or a combination thereof.

12. A method according to claim 10, wherein assessing the degree of the reflux comprises assessing a degree of a malfunction of a spermatic vein valves.

13. A method for assessing a degree of venous reflux to the prostate comprising:
(a) measuring testosterone concentration; and
(b) determining a degree of the reflux responsive to the testosterone concentration.

14. A method according to claim 13, wherein the testosterone comprises at least one of free or bound testosterone.

15. A method according to claim 13, wherein assessing the degree of the reflux is responsive to the anatomical location of the measurement.

16. A method according to claim 13, wherein assessing the degree of the reflux comprises assessing a degree of a malfunction of a spermatic vein valves.

17. A method according to claim 13, wherein the measurement comprises a measurement of at least one of a venous blood or arterial blood.

18. A method according to claim 17, wherein the measurement comprises a measurement in a blood vessel at an anatomical location near the prostate before the blood drain to the vena cava.

19. A method for reducing aging symptoms comprising effecting a cause that decreased testosterone supply to the blood stream.

20. A method according to claim 19, wherein effecting comprises impeding a reflux of venous blood.

21. A method according to claim 20, wherein impeding the reflux comprises treating veins effecting or conveying the reflux.

22. A method according to claim 21, wherein treating comprises occlusion.

23. A method according to claim 22, wherein occlusion comprises utilizing at least one of a sclerosant, an element that blocks the vein passage, an element that induces thrombosis, a material that induces thrombosis, ablation or medication.

24. A method according to claim 21, wherein the vein comprises an internal spermatic vein or a deferential vein or a by-pass vein.

25. A method of passing a wire or a catheter through a venous valve, comprising
(a) expanding a vein such that a valve orifice is opened; and
(b) moving the wire or catheter through the opened orifice.

26. A method according to claim 25, wherein the vein comprises one of an internal spermatic vein, a deferential vein or a by-pass vein.

27. A method according to claim 25, wherein expanding comprises utilizing an expandable element.

28. A method according to claim 27, wherein the expandable element is contractible.

29. A method according to claim 27, wherein the expandable element comprises a wire mesh.

30. A method according to claim 27, wherein the expandable element comprises an inflatable balloon.

31. A method according to claim 27, wherein the expandable element comprises an elastic element.

32. A guide-wire for passing a catheter through a venous valve comprising:
(a) an expandable element near a distal end of the guide-wire; and
(b) an extension at the distal end of the element, forming a flexible tip.

33. A guide-wire according to claim 32, wherein the expandable elements is contractible.

34. A guide-wire according to claim 32, wherein the guide-wire comprises a lumen.

35. A guide-wire according to claim 34, wherein a control wire passes through the lumen.

36. A guide-wire according to claim 35, wherein the control wire effects at least one of expanding or contracting the element.

37. A guide-wire according to claim 36, wherein
(a) pulling the control wire expands the element; and
(b) pushing the control wire retracts the element.

38. A guide-wire according to claim 32, wherein the element comprises a wire mesh.

39. A guide-wire according to claim 32, wherein the element comprises an inflatable balloon.

40. A guide-wire according to claim 32 wherein the element comprises an elastic element.

41. A guide-wire according to claim 32, wherein the element comprises a shape memory material (SMA) expanding or retracting responsive to temperature, or a piezoelectric element expanding or retracting responsive to voltage.

42. A vascular catheter for occluding a region of a blood vessel comprising:
(a) one or more inflatable elements; and
(b) one or more perforations along at least part of the catheter for a material application.

43. A catheter according to claim 42, wherein the material is a sclerosant injected into the catheter.

44. A catheter according to claim 42, wherein the material is a mechanical element removably attached to the catheter.

45. A catheter according to claim 42, wherein the length of an interval between the inflatable elements is modifiable.

46. A catheter according to claim 42, wherein the material is a glue.

47. A catheter according to claim 42, wherein the catheter applies electric field to induce sclerosis.

48. A catheter according to claim 42, wherein at least some of the perforations are closable.

49. A kit for forestalling or therapy, at least partially, of BPH or prostate cancer, comprising:
(a) a guide-wire for passing a catheter through a venous valve;
(b) a vascular catheter for occluding a region of a blood vessel; and
(c) an occlusion material or element.

50. A kit according to claim 49, wherein the material comprises at least one of a glue, a sclerosant, or an embolization material.

51. A kit according to claim 49, wherein the element is a coil or an elastic element.

52. A kit according to claim 49, comprising a plurality of the guide-wire or a plurality of the vascular catheters.

53. A kit according to claim 49, wherein the material or elements are sufficient for at least one treatment.

54. A kit according to claim 49, comprising an auxiliary device.

55. A kit according to claim 54, wherein the auxiliary device comprises one of a catheter port, a syringe, or other devices used with the guide-wire or the catheter or the occlusion material or the occlusion element.

56. Use of a sclerosant for the manufacture of medicament for treating BPH or prostate cancer in a subject.

57. A method according to claim 56, wherein said subject age is about 40 or over, 50 or over or 60 or older.

58. A method according to claim 56, wherein said subject was not diagnosed as infertile or not diagnosed for varicocele.
